(19) United States
(12) Patent Application Publication
(10) Pub. No.: US 2026/0232200 A1
(43) Pub. Date: Aug. 13, 2026

(54) Method of assessing weight of an infant and the device thereof

(71) Applicant: N.V. Nutricia, Zoetermeer (NL)

(72) Inventors: Sankha Mukherjee, Singapore (SG); Agathe Camille Foussat, Singapore (SG); Jill Wong, Singapore (SG); Matthew Hadimaja, Singapore (SG); Umesh Nandal, Singapore (SG)

(73) Assignee: N.V. Nutricia, Zoetermeer (NL)

(21) Appl. No.: 19/543,947

(22) Filed: Feb. 19, 2026

Related U.S. Application Data
(63) Continuation of application No. PCT/EP2023/073528, filed on Aug. 28, 2023.

(51) Int. Cl. A61B 5/00; A61B 5/107; G01G 19/44; G01G 23/35; G06T 7/00

(52) U.S. Cl. CPC A61B 5/0077; A61B 5/1072; A61B 5/1079; A61B 5/7275; G01G 19/44; G01G 23/35; G06T 7/0012; A61B 2503/04; G06T 2207/20081; G06T 2207/20084; G06T 2207/30196

(57) ABSTRACT

A method performed by an electronic device assessing weight of an infant, obtaining visual information including the infant; obtaining basic information of the infant; determining size information of the infant based on the visual information, the size information comprising at least one of length information of the infant, face information of the infant, pose information of the infant, body shape information of the infant, and chubbiness information of the infant; determining the weight of the infant based on the size information and the basic information.

### FIELD OF THE INVENTION

The present invention relates to a method of assessing weight of an infant, and an apparatus and a computer-implemented algorithm thereof. More specifically, the method of assessing weight of an infant may be based on visual information of the infant and a reference object. In addition, the present invention relates to a method of comparing the growth trajectory of an infant fed with one or more nutritional compositions, with the growth trajectory of an infant fed with one or more other nutritional compositions, e.g., human milk.

### BACKGROUND

The healthy development of babies is always the most important concern to parents and caretakers. It is a general desire for parents and caretakers to assess the development of an infant as easily as possible.

Generally, breastfeeding is preferred for babies to acquire suitable nutrients. However, in some cases, breastfeeding is not suitable due to medical reasons or other reasons and an infant formula is used. As a result, it frequently happens that parents visit the paediatrician or healthcare provider (HCP) more than necessary to track the growth trajectory of their baby when their baby is fed with an infant formula, because of their concerns about whether their baby has taken enough nutrients.

Nowadays, infant weight measurements are mostly via the use of professionally calibrated weighing scales with trained personnel. However, infants or young children usually keep moving and seldom stay still when they are awake, and it is difficult to obtain an accurate weight. In addition, seeking help from professionals is costly and time-consuming. Although parents can measure the weight by themselves, either special equipment is needed, and/or, it can still be time consuming. Not to mentioned how difficult it can be to keep the record and see the trend from a term. As a result, many parents may give up recording the growth trajectory of their babies after a while.

However, weight is a very important indicator of the baby's growth and may comparable growth information of the babies under the same condition or even predict health-related issues of the babies where early intervention may be needed, especially for babies between the ages of 0 and 2 years and for comparison of babies fed by human milk.

To allow parents and caretakers to track the development of their babies, WO2017/099580 A1 discloses a system for tracking infant development by using infant weight information provided by measuring devices such as a weighing scale and information derived from a camera image of the infant captured by a portable device. However, it may still be inconvenient due to the multiple steps to be performed and multiple devices they are used.

There are also some systems that predict the body mass index (BMI) of individuals from facial features, where BMI is a person's weight in kilograms divided by the square of height in meters. A high BMI can indicate high body fatness. Hera Siddiqui, et al., AI-based BMI Inference from Facial Images: An Application to Weight Monitoring, discloses an AI-based method of calculating BMI for adults from facial features. However, estimating body weight merely based on facial features sometimes can be inaccurate. For example, some people may seem to be chubby on their faces but they actually have thin bodies, and vice versa. As a result, such systems may be used to coarsely get a sense of a person's body weight, but is not rigorous to monitor the weight changes of an infant. Furthermore, the babies have totally different body shape than adults, thus, the general method from Hera Siddiqui, et al. for adult weight measurement may not be suitable for babies.

Therefore, there is a need for a method of assessing the weight of an infant in a convenient, and accurate manner for parents who would like to keep close track of the growth trajectory of their babies, especially for babies from 0 to 2 years.

### SUMMARY OF THE INVENTION

In order to solve the above technical problem, the present invention provides a method of assessing weight of an infant, and an apparatus and a computer-implemented algorithm thereof.

The present invention provides a method for assessing weight of an infant via visual information, e.g., two-dimensional, or three-dimensional information which may be obtained from a camera, received from externally, or prestored. As an example of the present invention the weight of the baby may be determined based on a picture of the baby, along with a reference object (for example, a credit card or a ruler) placed close to the baby within the same picture frame. Additional basic information of the babies may be used as well when determining the weight. The present invention allows parents to monitor the growth of their children in a more consistent manner and thus allows early intervention for abnormal growth if necessary.

The present invention further aims to: lower the effort from the parents/caregivers to enter the weight of a baby on a regular basis, allowing apps to capture regular measurements of the weight of the baby; allow downstream processing of weights for a baby allowing for early detection of whether a baby is over/underweight or on track on their growth trajectories; and allow early intervention in case of babies with abnormal growth trajectories.

The present invention provides a method for comparing the growth trajectory of an infant fed with one nutritional composition, e.g., an infant formula, with the growth trajectory of an infant fed with another nutritional composition, e.g., human milk or another infant formula.

The present invention can also provide an easy way of monitoring population-based improvements in its early-life milk products and providing suggestions for early nutritional supplemental interventions to individuals in need. This is especially true in some less developed countries, where the medical system has a paucity of doctors, and nutritional knowledge for early life is lacking.

The present invention further provides an electronic device for assessing weight of an infant. The electronic device comprises an input module and a processor. The electronic device is configured to: obtain, by the camera, a visual information of the infant; obtain, by the input module, basic information of the infant; determine, by the processor, size information of the infant based on the visual information, the size information comprising at least one of length information of the infant, face information of the infant, pose information of the infant, body shape information of the infant, and chubbiness information of the infant; determine, by the processor, the weight of the infant based on the size information and the basic information.

In the present invention, the determining of the size information and the determining of the weight are based on trained AI models or mapping tables. Data for these trained AI models or mapping tables may be obtained from hospitals and research institutions for the measurement of both weight and length.

Other aspects, advantages, and salient features of the disclosure will become apparent to those skilled in the art from the following detailed description, which, taken in conjunction with the annexed drawings, discloses various embodiments of the present disclosure.

### BRIEF DESCRIPTION OF THE DRAWINGS

The present invention will be discussed in more detail below, with reference to the attached drawings, in which:

FIG. 1 shows a method of determining weight of an infant.

FIG. 2 shows an example of visual information including an infant.

FIG. 3 shows an example of face information.

FIG. 4 shows an example of pose information.

FIG. 5 shows an example of body shape information.

FIG. 6 shows an example of a growth trajectory of an infant based on the weight and basic information.

FIG. 7 shows a method of comparing a growth trajectory of an infant fed by a nutritional composition with a growth trajectory of another infant fed by another nutritional composition.

FIG. 8 shows an example of a comparison of the growth trajectory of an infant fed by an infant formula with a growth trajectory of another infant fed by human milk.

FIG. 9 shows an electronic device.

### DESCRIPTION OF EMBODIMENTS

The present invention preferably provides a method and a device thereof to assess weight of an infant based on visual information of an infant.

Terms used in the present disclosure are used for describing particular embodiments and are not intended to limit the scope of other embodiments. A singular form may include a plurality of forms unless it is explicitly differently represented. All the terms used herein, including technical and scientific terms, may have the same meanings as terms generally understood by those skilled in the art to which the present disclosure pertains.

In the present disclosure disclosed herein, the expressions “A or B”, “at least one of A or/and B”, “A, B, or C” or “one or more of A, B, or/and C”, and the like used herein may include any and all combinations of one or more of the associated listed items. The terms “include” and “comprise,” as well as derivatives thereof, mean inclusion without limitation.

The term “module” used herein may represent, for example, a unit including one or more combinations of hardware, software and firmware. The term “module” may be interchangeably used with the term “unit”. The “module” may be a minimum unit of an integrated part or may be a part thereof. The “module” may be a minimum unit for performing one or more functions or a part thereof.

The present application provides a method performed by an electronic device for assessing weight of an infant. The method may comprise: obtaining visual information including the infant; obtaining basic information of the infant; determining size information of the infant based on the visual information; and determining the weight of the infant based on the size information and the basic information. The size information may comprise at least one of length information of the infant, face information of the infant, pose information of the infant, body shape information of the infant, and chubbiness information of the infant.

The determining of the size information of the infant may be based on a reference object in the visual information, and the reference object may be a card or a ruler.

The basic information may comprise at least one of gender, birth weight, and age of the infant. The chubbiness features may include at least one of head circumference, skinfold thickness, and a size of waist and bust of the infant. The skinfold thickness may include at least one of thickness of, bicep folds, triceps folds, suprailiac folds and scapular lower folds, of the infant. The face information may include at least one of left eyebrow width, right eyebrow width, left eye width, right eye width, nose width, nose length, outer lid width, inner lip width, face height, and face width. The pose information may include at least one of: the length of left-shoulder to left-hip; the length of right-should to right-hip; the length of left-hip to right-hip; the length of left-shoulder to right-hip; the length of right-shoulder to left-hip; the length of left-shoulder to left-elbow; the length of left-elbow to left-wrist; the length of right-shoulder to right-elbow; and the length of right-elbow to right-wrist. The body shape information of the infant may be determined based on at least one of waist width, hip width, and thigh width.

The determining of the size information may be performed by a first trained AI model, and/or the determining of the weight may be performed by a second trained AI model or a pre-determined table.

Each of the size information may be determined in a separate AI model.

The method may further include determining the growth trajectory of the infant based on the weight and the basic information. The determined growth trajectory may comprise a growth score, and the growth score may be obtained by a comparison of the determined growth trajectory with a reference growth trajectory.

The method may further include comparing the growth trajectory of the infant fed by a nutritional composition with another growth trajectory of another infant fed by another nutritional composition and determining a similarity level between the two growth trajectories.

The determining of the growth trajectory may further be based on face expression of the infant, and the face expression may be determined based on the visual information.

The nutritional composition may be an infant formula or follow-on formula and the another nutritional composition may be human milk.

The infant may be between 0 and 2 years old.

The present application provides an electronic device for assessing weight of an infant. The electronic device may comprise: an input module and a processor. The electronic device may be configured to: obtain visual information of the infant; obtain, by the input module, basic information of the infant; determine, by the processor, size information of the infant based on the visual information, and determine, by the processor, the weight of the infant based on the size information and the basic information. The size information may comprise at least one of length information of the infant, face information of the infant, pose information of the infant, body shape information of the infant, and chubbiness information of the infant.

The electronic device may further be configured to determine, by the processor, a growth trajectory of the infant based on the weight and the basic information.

The electronic device may further be configured to: compare, by the processor, the growth trajectory of the infant fed by a nutritional composition to another growth trajectory of another infant fed by another nutritional composition; and determine, by the processor, a similarity level between the two growth trajectories.

The electronic device may further comprise a storage unit, and the storage unit may be configured to store the basic information of the infant and programs and/or instructions for the determining of the size information of the infant and the determining of the weight of the infant.

The storage unit may further be configured to store programs and/or instructions for the determining of the growth trajectory of the infant, the comparing of the growth trajectory of the infant fed by the nutritional composition to the another growth trajectory of the another infant fed by the another nutritional composition; and the determining of the similarity level between the two growth trajectories.

The electronic device may comprise a display. The display may be configured to display the visual information and the growth trajectory.

The present application provides a storage medium. The storage medium may comprise program instructions which, when executed on an electronic device, cause the electronic device to perform the method mentioned above for assessing weight of an infant.

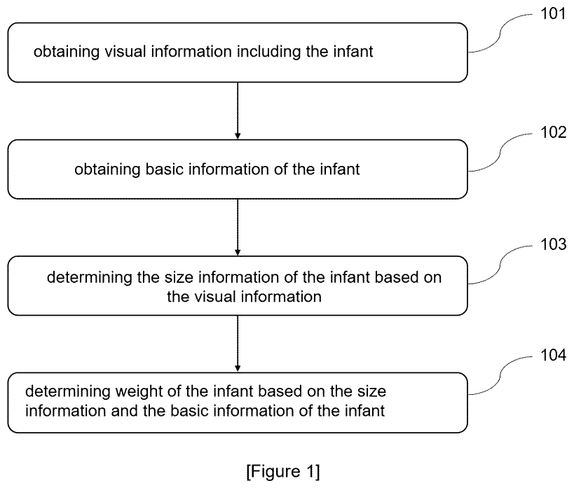

FIG. 1 shows a method of determining weight of an infant.

In step 101, visual information including the infant is obtained. The visual information may be two-dimensional or three-dimensional visual information, e.g., images captured by a camera, three-dimensional visual information obtained by a 3D scanner, depth information from a Light Detection and Ranging (liDAR) system or sensors, etc. The visual information may also be received from an external device or server, or prestored in a database. In this document, the term “image” is used interchangeably with the term “visual information”.

If a camera is used to obtain the visual information, the camera may be a camera of an electronic device, such as a mobile phone, a tablet, a laptop, etc.

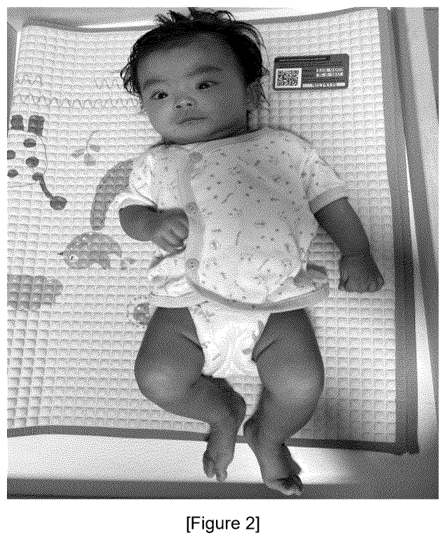

FIG. 2 shows an example of obtained visual information. When an image of an infant is captured with a camera, a reference object may be included in the same image. Preferably, when capturing the image of an infant, the infant is facing the camera. More preferably, the infant is wearing tight-fitting clothes such as onesies instead of loose-fitting clothes such as loose dresses and wraps.

Furthermore, the reference object in the image may be clearly visible and close to the infant. Preferably, the reference object is not partially or wholly covered by the infant. The reference object may be a ruler or a card, such as a credit card or a debit card, which may be used to determine the size information of the infant in step 103 which will be explained later in this document, based on size information of the reference object. The size information of reference object may be inputted by the user or prestored in a memory or a database locally or remotely. For example, the size information of the reference object may be inputted via an input device, such as a keyboard, a microphone, or part of the display of a mobile phone, a tablet, or a laptop, etc. Or, the prestored size information of the reference object may be stored in a storage unit in the electronic device or may be stored in a database on a remote server.

In step 102, basic information of the infant is obtained.

The basic information of the infant may comprise at least one of gender, birth weight, and age of the infant. The basic information may be inputted by the user or prestored in a memory or a database. For example, the basic information may be inputted via an input device, such as a keyboard, a microphone, or part of the display of a mobile phone, a tablet, or a laptop, etc. Or, the prestored basic information may be stored in a storage unit in the electronic device or may be stored in a database on a remote server.

In step 103, the size information of the infant is determined based on the visual information.

The size information of the infant may comprise at least one of length information of the infant, face information of the infant, pose information of the infant, body shape information of the infant, and chubbiness information of the infant.

The length information of the infant may comprise at least one of the height of the infant and the width of the infant. The width of the infant is the distance between the two hands when the arms are fully outstretched at the sides.

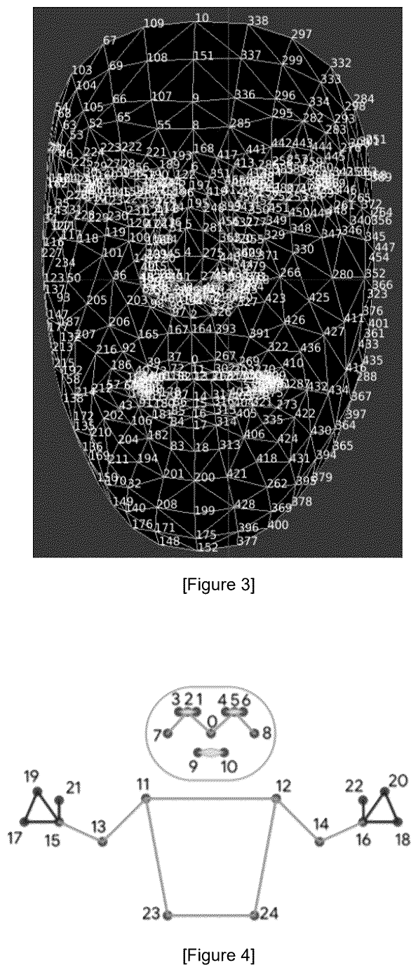

The face information may include at least one of left eyebrow width (18-22), right eyebrow width (23-27), left eye width (37-40), right eye width (43-46), nose width (32-36), nose length (28-34), outer lid width (49-55), inner lip width (61-65), face height (28-9), and face width (1-17), as shown in FIG. 3.

The pose information of the infant may include the lengths of body parts of the infant. For example, in FIG. 4, the lengths of the body parts may include at least one of: the length of left-shoulder to left-hip (11-23); the length of right-should to right-hip (12-24); the length of left-hip to right-hip (23-24); the length of left-shoulder to right-hip (11-24); the length of right-shoulder to left-hip (12-23); the length of left-shoulder to left-elbow (11-13); the length of left-elbow to left-wrist (13-15); the length of right-shoulder to right-elbow (12-14); and the length of right-elbow to right-wrist (14-16).

The body shape information of an infant may include the area of body segmentation of the infant in the image, the ratio of waist width to hip width, the ratio of hip width to face width, the ratio of waist width to thigh width, and the ratio of waist width to face width. The area of the body segmentation of the infant is determined based on the above various widths of the body parts of the infant in the pose information. For example, the area of the body segmentation of the infant may be determined based on the waist width and thigh width of the infant.

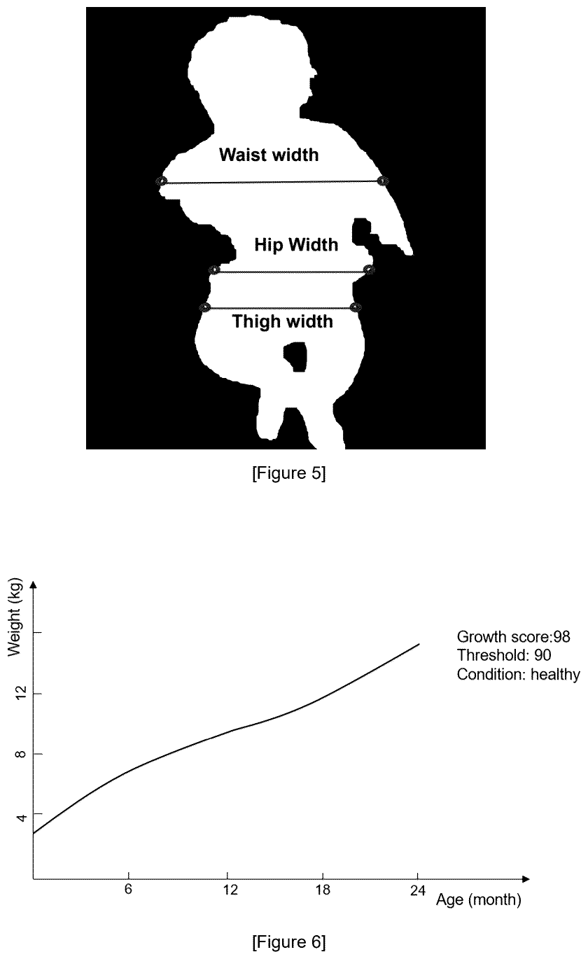

FIG. 5 shows an example of an image of an lying down infant. The area of body segmentation can be determined according to Equation 1, as mentioned below:

$$Area = \frac{\#pixels}{( {y}_{h} - {y}_{w} ) \cdot 0.5 \cdot [ d ( {p}_{lwb} , {p}_{rwb} ) + d ( {p}_{lhb} , {p}_{rhb} ) ]} , \qquad Equation 1$$

wherein #pixels represents the number of the pixels included in the body segmentation area in the image, yₕ represents the hip width of the infant, y_{w} represents the waist width of the infant, p_{lwb} represents the pixel of the left waist boundary, p_{rwb} represents the pixel of the right waist boundary, p_{lnb} represents the pixel of the left hip boundary, p_{rhb} represents the pixel of the right hip boundary, and d(p_{lwb}, p_{rwb}) is the distance between the pixel p_{lwb} and the pixel p_{rwb}, and d(p_{lnb}, p_{rhb}) is the distance between the pixel p_{lnb} and the pixel p_{rhb}. The distance measure d(·,·) may be Euclidean distance.

The ratio of waist width to hip width is determined according to Equation 2, as mentioned below.

$$whr = \frac{waist width}{hip width} \qquad Equation 2$$

The ratio of hip width to face width is determined according to Equation 3, as mentioned below.

$$hfr = \frac{hip width}{face width} \qquad Equation 3$$

The ratio of waist width to thigh width is determined according to Equation 4, as mentioned below.

$$wtr = \frac{waist width}{thigh width} \qquad Equation 4$$

The ratio of waist width to face width is determined according to Equation 5, as mentioned below.

$$wtr = \frac{waist width}{thigh width} \qquad Equation 5$$

The chubbiness information of the infant may comprise at least one of head circumference, skinfold thickness, and a size of waist and bust of the infant. The head circumference, also referred to as OFC (occipital frontal circumference) may be measured over the most prominent part of the back of the head, known as the occiput, and above the eyebrows, known as the supraorbital ridges, conforming the largest circumference of the head. The head circumference may be determined by using image processing techniques by a processor based on the visual information. For example, to determine the head circumference, at least two images or a movie may be taken of the infant from different positions, such as from the front of the baby's head, from a side, and from the backside. The skinfold thickness is a non-invasive measurement of the body fat of an infant and is indicative of physical development. The skinfold thickness comprises at least one of bicep folds thickness, triceps folds thickness, suprailiac folds thickness, and scapular lower folds thickness. Preferably, the skinfold thickness is subscapular skinfold thickness and/or triceps skinfold thickness.

The determining of the size information of the infant may be performed by at least one first trained AI model. The at least one first trained AI model has been trained with available infant data. The infant data may be obtained from hospitals and research institutions for the measurement of both weight and length. Once the first AI model is trained, the first trained AI model may output size information based on the input of visual information of the infant (e.g., infant images). The first trained AI model may be a model that is able to identify an object length in an image based on the reference object. For example, the first trained AI model may comprise at least one of an analytic reconstruction model, an iterative reconstruction model, a lidar reconstruction model, a three-dimensional reconstruction model, preferably the at least one reconstruction model is the lidar reconstruction model.

When determining the size information of the infant, each of the size information may be determined in a separate AI model, or all the size information may be determined in the same AI model, or some of the size information of the infant may be determined in different AI models and some of the size information of the infant may be determined in one same AI model.

In step 104, weight of the infant is determined based on the size information and the basic information of the infant.

The determining of weight of the infant may be performed by a second trained AI model or via a pre-determined mapping table.

In case that at least one second trained AI model is used to determine the weight of the infant, the at least one second trained AI model may be a deep learning model that maps the size information and the basic information of an infant to the weight of the infant. The second AI model may be trained on available infant data. The infant data may be obtained from hospitals and research institutions for the measurement of both weight and length. Once the second AI model is trained, the second trained AI model may output the weight of the infant with inputs of the size information of the infant (e.g., at least one of length information of the infant, face information of the infant, pose information of the infant, body shape information of the infant, and chubbiness information of the infant) and the basic information of the infant (e.g., at least one of gender, birth weight, and age of the infant).

An example of calculating the weight of an infant is as follows. First, the length of an infant may be calculated by using the Length AI algorithm, I_{LAI}. Then, the age of the infant may be converted into days if the age of the infant provided is counted by weeks or months. The months may be converted into days by Equation 6 and the weeks may be converted into days by Equation 7, as mentioned below.

$${f}_{mtd} ( m ) = ( m + 0.5 ) \times \frac{365}{12} \qquad Equation 6$$

$${f}_{wtd} ( w ) = ( w + 0.5 ) \times 7 \qquad Equation 7$$

wherein w represents the age in weeks and m represents the age in months, ƒ_{mtd}(m) represents the age in days converted from months, and ƒ_{wtd}(w) represents the age in days converted from weeks.

The weight of the infant may be calculated by Equation 8, as mentioned below.

$$w ( a , g ) = f ( p ( a , g ) ; {l}_{LAI} \vert {w}_{NN} ) \qquad Equation 8$$

wherein a represents the age of an infant in days, g represents the gender of the infant, either male or female, p(a, g)={pⁱ(a, g)|i∈[0, 1, 3, 5, 10, 15, 25, 50, 75, 85, 97, 99, 100]} represents a vector or interpolated percentiles, and the function ƒ is a learnable parametric model (e.g., a neural network) whose parameters (w_{NN}) learned from training data. p(a, g) may be obtained from data charts provided by at least one authority, e.g., The World Health Organization (WHO).

In case that a predetermined mapping table may be used to determine the weight of the infant, the predetermined mapping table may be constructed with multiple entries, and each of the entries includes the weight of infants, size information of infants, and basic information of infants. Given the size information of an infant (e.g., at least one of length information of the infant, face information of the infant, pose information of the infant, body shape information of the infant, and chubbiness information of the infant) and the basic information of the infant (e.g., at least one of nationality, gender, birth weight, and age of the infant), the corresponding weight may be looked up in the table.

The first trained AI model and the second trained AI model may be retrained when new data is available to obtain more accurate information.

The method of assessing weight of an infant further may comprise determining the growth trajectory of the infant based on the weight and the basic information. The growth trajectory may indicate, but is not limited to, the weight variation as an infant's age changes, the average weight variation of infants as the infants' age change, etc. Preferably, the method of assessing weight of an infant further comprises determining a growth score of the growth trajectory. The growth score may be obtained a comparison of the determined growth trajectory with a reference growth trajectory. In case the growth score is equal to or greater than a threshold, the growth of the infant is similar to the reference growth trajectory, which indicates the infant is healthy. Otherwise, the growth of the infant is not similar to the reference growth trajectory, which indicates the infant is unhealthy.

More preferably, the determining of the growth trajectory may be further based on face expression of the infant. For example, the determining of the growth trajectory may be further based on whether the infant has face expressions such as smiling, laughing, surprising, etc, because the advances in face expressions can give indications for infant growth as well. The face expression is determined based on the visual information.

FIG. 6 shows an example of a growth trajectory of an infant determined based on the weight and the basic information. In FIG. 6, the X-axis represents the age (month) of the infant and the Y-axis represents the weight (kg) determined in step 104. The growth score of the growth trajectory is 98 and the threshold is 90. Since the growth score of the growth trajectory is greater than the threshold, the infant is healthy.

The growth trajectories may be used to evaluate the development of infants. For example, a growth trajectory of an infant fed by a nutritional composition may be compared with a growth trajectory of another infant fed by another nutritional composition (e.g., the nutritional composition is an infant formula or follow-on formula and the another nutritional composition is human milk). For another example, a growth trajectory of an infant may be compared with an average growth trajectory of infants (e.g., compared with an average growth trajectory of infants from the same country where the infant is born). For another example, an average growth trajectory of infants from one geographic region may be compared with an average growth trajectory of infants from another geographic region.

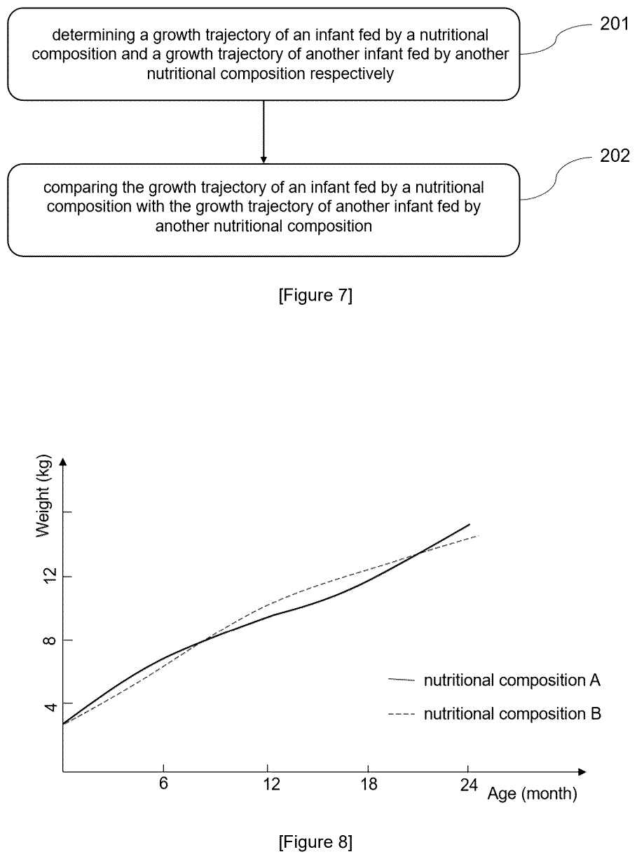

FIG. 7 shows a method of comparing a growth trajectory of an infant fed by a nutritional composition with a growth trajectory of another infant fed by another nutritional composition.

In step 201, determining a growth trajectory of an infant fed by a nutritional composition and a growth trajectory of another infant fed by another nutritional composition respectively.

FIG. 8 shows an example of a comparison of the growth trajectory of an infant fed by an infant formula with a growth trajectory of another infant fed by human milk. In FIG. 8, the X-axis represents the age (month) of the infant and the Y-axis represents the weight (kg).

In step 202, comparing the growth trajectory of an infant fed by a nutritional composition with the growth trajectory of another infant fed by another nutritional composition. The comparing of the two growth trajectories may include determining a similarity level between the two growth trajectories. The similarity level may be determined based on the at least one result of the comparison and at least one corresponding predetermined threshold. If the at least one result of the comparison is greater than or equal to at least one corresponding predetermined threshold, the two growth trajectories have a high similarity level, which means the two growth trajectories are similar to each other. If the at least one result of the comparison is smaller than at least one corresponding predetermined threshold, the two growth trajectories have a low similarity level, which means the two growth trajectories are not similar to each other.

To be mentioned, although there are only two growth trajectories in the examples, more growth trajectories may be included, e.g., based on a third nutritional composition fed to a third infant.

The present invention further provides an electronic device to perform the method above.

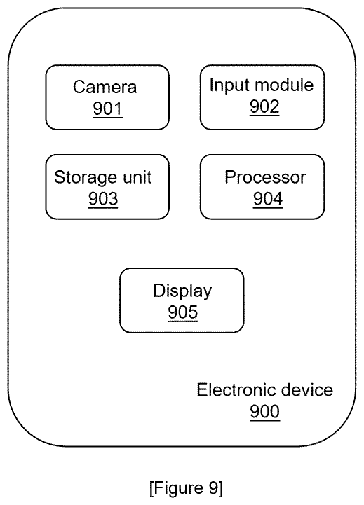

FIG. 9 shows a block diagram of an example electronic device according to the embodiment of the present invention.

The electronic device 900 comprises a camera 901, an input module 902, a storage unit 903, a processor 904, and a display 905. The electronic device 900 may be a mobile phone, a tablet, a laptop, etc.

The camera 901 may be used to capture a still image or a video of an object. For example, the camera 901 may be used to obtain visual information in which an infant and a reference object may be included.

The input module 902 may be used to input basic information of infants by users. For example, the input module 902 may be a keyboard, a microphone, a touch screen, etc.

The storage unit 903 may store basic information of infants including gender, and age, etc., and programs and/or instructions for determining the size information of the infant and determining the weight of the infant. Optionally, the storage unit 903 may further store programs and/or instructions for determining the growth trajectory of the infant, comparing the growth trajectory of the infant fed by a nutritional composition to another growth trajectory of another infant fed by another nutritional composition; and determining a similarity level between the two growth trajectories. The storage unit 903 may include a volatile memory, a non-volatile memory, or a combination of a volatile memory and a non-volatile memory. The storage unit 903 may be provided in the electronic device. Alternatively or additionally, the storage unit 903 may be provided on a server connected via a network such as the Internet (e.g. cloud storage). The storage unit 903 may be a cumulative storage, where data is added with each new measurement. The storage unit 903 thus may include all measurements data.

The processor 904 may determine the size information of the infant based on the visual information obtained by the camera 901. The processor may determine the weight of the infant based on the size information and the basic information inputted from the input module 902 or stored in the storage unit 903. The processor may determine the growth trajectory of the infant based on the weight and the basic information and control a display 905 to display the determined growth trajectory.

The input module 902 and the display 905 may be separate components of the electronic device. The input module 902 and the display 905 may be integrated. For example, the input module 902 may be part of the display 905.

The present invention relates to a storage medium comprising program instructions to instruct an electronic device to perform any of the methods mentioned above.

Meanwhile, while the specific embodiment has been described in the explanations of the present disclosure, it will be noted that various changes may be made therein without departing from the scope of the disclosure. Thus, the scope of the disclosure is not limited and defined by the described embodiment and is defined the scope of the claims.

1. A method performed by an electronic device for assessing weight of an infant, comprising:
obtaining visual information including the infant;
obtaining basic information of the infant;
determining size information of the infant based on the visual information, the size information comprising at least one of length information of the infant, face information of the infant, pose information of the infant, body shape information of the infant, and chubbiness information of the infant;
determining the weight of the infant based on the size information and the basic information.

2. The method according to claim 1, wherein the determining of the size information of the infant is based on a reference object in the visual information.

3. The method according to claim 1, wherein the reference object is a card or a ruler.

4. The method according to claim 1, wherein the basic information comprises at least one of gender, birth weight, and age of the infant.

5. The method according to claim 1, wherein the chubbiness features include at least one of head circumference, skinfold thickness, and a size of waist and bust of the infant, wherein the skinfold thickness includes at least one of thickness of, bicep folds, triceps folds, suprailiac folds and scapular lower folds, of the infant.

6. The method according to claim 1, the face information includes at least one of: left eyebrow width, right eyebrow width, left eye width, right eye width, nose width, nose length, outer lid width, inner lip width, face height, and face width.

7. The method according to claim 1, the pose information includes at least one of: the length of left-shoulder to left-hip; the length of right-should to right-hip; the length of left-hip to right-hip; the length of left-shoulder to right-hip; the length of right-shoulder to left-hip; the length of left-shoulder to left-elbow; the length of left-elbow to left-wrist; the length of right-shoulder to right-elbow; and the length of right-elbow to right-wrist.

8. The method according to claim 1, wherein the body shape information of the infant is determined based on at least one of waist width, hip width, and thigh width.

9. The method according to claim 1, the determining of the size information is performed by a first trained AI model, and/or the determining of the weight is performed by a second trained AI model or a pre-determined table.

10. The method according to claim 1, wherein each of the size information is determined in a separate AI model.

11. The method according to claim 1, wherein the method further includes determining the growth trajectory of the infant based on the weight and the basic information.

12. The method according to claim 11, wherein the determined growth trajectory comprises a growth score, wherein the growth score is obtained by a comparison of the determined growth trajectory with a reference growth trajectory.

13. The method according to claim 11, the method further includes:
comparing the growth trajectory of the infant fed by a nutritional composition with another growth trajectory of another infant fed by another nutritional composition; and
determining a similarity level between the two growth trajectories.

14. The method according to claim 11, the determining of the growth trajectory is further based on face expression of the infant, wherein the face expression is determined based on the visual information.

15. The method according to claim 13, wherein the nutritional composition is an infant formula or follow-on formula and the another nutritional composition is human milk.

16. The method according to claim 1, wherein the infant is between 0 and 2 years old.

17. An electronic device for assessing weight of an infant, comprising:
an input module; and
a processor, and
wherein the electronic device is configured to:
obtain visual information of the infant;
obtain, by the input module, basic information of the infant;
determine, by the processor, size information of the infant based on the visual information, the size information comprising at least one of length information of the infant, face information of the infant, pose information of the infant, body shape information of the infant, and chubbiness information of the infant;
determine, by the processor, the weight of the infant based on the size information and the basic information.

18. The electronic device according to claim 17, wherein the electronic device is further configured to determine, by the processor, a growth trajectory of the infant based on the weight and the basic information.

19. The electronic device of claim 18, wherein the electronic device is further configured to:
compare, by the processor, the growth trajectory of the infant fed by a nutritional composition to another growth trajectory of another infant fed by another nutritional composition; and
determine, by the processor, a similarity level between the two growth trajectories.

20. The electronic device according to claim 17, further comprises a storage unit, and the storage unit is configured to store the basic information of the infant and programs and/or instructions for the determining of the size information of the infant and the determining of the weight of the infant.

21.-23. (canceled)
